(19) United States
(12) Patent Application Publication
(10) Pub. No.: US 2009/0081077 A1
(43) Pub. Date: Mar. 26, 2009

(54) ALKALINE WATER STERILIZER AND ALKALINE STERILIZING WATER PRODUCTION METHOD

(75) Inventor: Yuichi SAWADA, Shimane (JP)

Correspondence Address: YOUNG & THOMPSON, 209 Madison Street, Suite 500, ALEXANDRIA, VA, 22314, US

(73) Assignee: KINJI SAWADA, TOKYO (JP)

(21) Appl. No.: 11/965,803

(22) Filed: Dec. 28, 2007

(30) Foreign Application Priority Data
Dec. 28, 2006 (JP) 2006-346650

(51) Int. Cl. A61L 2/03

(52) U.S. Cl. 422/37; 205/742

(57) ABSTRACT

This invention, in addition to both more adequate disinfection effect and more adequate cleaning effect, offers a better electrolyzed alkaline water production unit and a better electrolyzed alkaline water production method in terms of storage stability before use or safety in comparison with strong alkaline solution.

An alkaline water sterilizer 100 produces electrolyzed alkaline water by electrolyzing tap water which is neutral water by an electrolyzed alkaline water production unit 100a. Then, the electrolyzed alkaline water thus produced is electrolyzed by a hypochlorite water production unit 100b to produce hypochlorous acid by electrolysis and hypochlorite water having a pH value of at least 10 or more is produced as alkaline sterilizing water.

### BACKGROUND OF THE INVENTION

1. Field of the Invention

The present invention relates to an alkaline water sterilizer and an alkaline sterilizing water production method.

2. Discussion of the Related Art

As a production apparatus of electrolyzed water, various types are known. For example, in Patent Document 1, a batch type electrolyzed water production apparatus which collects produced electrolyzed water is disclosed.

Moreover, in Patent Document 2, an electrolyzed water production apparatus which produces sterilizing water by further electrolyzing unused electrolyzed water is disclosed.

In addition, in Patent Document 3, an electrolyzed water production apparatus which neutralizes alkaline water by way of neutralizer and returns the water into a stock water tank for the purpose of obtaining acid water is described.

Furthermore, in Patent Document 4, an electrolyzed water production apparatus which can produce either regenerated water or acid water efficiently because the apparatus includes a regenerated water generation room and an acid water generation room, each room has a stock water inlet and electrolyzed water outlet, the regenerated water generation room and/or the acid water generation room can include a circulation system for circulating either regenerated water or acid water which is not used and by including such a circulation system, either of the regenerated water or the acid water can be collected for use while the other is circulated is disclosed.

Furthermore, in recent years, it has been known that electrolyzed water obtained by electrolyzing various kinds of solution has sterilizing effect and such electrolyzed sterilizing water is applied to various types of sterilization and antisepsis. As a conventional production method of electrolyzed sterilizing water, for example, as in Patent Document 5, water to which sodium chloride has been added is transferred to an electrolyzer including a diaphragm to be electrolyzed so that strong acid water generated on anode side is obtained as electrolyzed sterilizing water is known. Moreover, in a technology disclosed in a specification of Patent Document 6, a method by which water to which sodium chloride has been added and water to which hydrochloric acid has been added are mixed together to be electrolyzed in a non-diaphragm electrolyzer is disclosed. In this document, water to which sodium chloride has been added is necessary additive to increase efficiency when electrolyzing the water. In addition, in Patent Documents 7 and 8, a method by which hydrochloric acid solution which substantially does not include sodium chloride is transferred to a non-diaphragm electrolyzer to be electrolyzed so that electrolyzed sterilizing water is obtained is disclosed.

Because the electrolyzed sterilizing water made by these techniques has higher sterilizing effect than, for example, chlorine water prepared by solving sodium hypochlorite in water and, in addition, because it is not required to adjust concentration every time the sterilizing water is used, it is regarded to be suitable as disinfectant.

Among the related arts, in Patent Document 4, there is a description that the electrolyzed alkaline water has sterilizing effect and detergent effect.

### [Patent Document 1] Japanese Published Unexamined Patent Application No. 2001-070941

### [Patent Document 2] Japanese Published Unexamined Patent Application No. 2001-062453

### [Patent Document 3] Japanese Published Unexamined Patent Application No. 2001-288545

### [Patent Document 4] Japanese Published Unexamined Patent Application No. 2003-53345

[Patent Document 5] Japanese Published Unexamined Patent Application No. H1-180293

### [Patent Document 6] Japanese Patent Specification No. 2627100

### [Patent Document 7] Japanese Patent Application No H8-309920

[Patent Document 8] Japanese Patent Application No. H10-189744

### SUMMARY OF THE INVENTION

However, as a result of reviewing by the inventor of the present invention, it was found out that there may be a case where the alkaline regenerated water according to Patent Document 4 cannot obtain enough sterilizing effect or detergent effect.

As a recent report says that pH of alkali hardly is related to sterilizing effect, according to a reviewing by the inventor of the present invention, there is a case where a large pH value does not mean enough sterilizing effect.

Moreover, in a method by which hypochlorite electrolysis product which generates hypochlorous acid by electrolyzing sodium chloride or the like is electrolyzed to produce electrolyzed sterilizing water, while sterilizing effect can be acquired by hypochlorous acid, alkaline property is not sufficient enough and there may be a case where detergent effect is not sufficient enough.

In addition, a method by which hypochlorite electrolysis product is added to strong alkaline solution already having a large enough pH value to generate hypochlorous acid was reviewed by the inventor or the present invention. However, there may be a case where using strong alkaline solution causes a problem from a viewpoint of storage stability before use or safety.

The present invention has been made in consideration of the above problems and is mainly aimed at providing an alkaline water sterilizer and a production method of alkaline sterilizing water which has both enough sterilizing effect and enough detergent effect and, in addition, has better storage stability and safety when compared with strong alkaline solution.

### MEANS FOR SOLVING PROBLEM

A feature of the present invention is an alkaline water sterilizer including an electrolyzed alkaline water production unit for producing electrolyzed alkaline water by electrolyzing neutral water and a hypochlorite water production unit for producing hypochlorite water of which pH is 10 or more by electrolyzing the electrolyzed alkaline water in which hypochlorite electrolysis product which generates hypochlorous acid by electrolysis is solved.

A feature of the present invention is an alkaline water sterilizer including a hypochlorite water production unit for producing hypochlorite water by electrolyzing neutral water in which hypochlorite electrolysis product which generates hypochlorous acid by electrolysis is solved and an electrolyzed alkaline water production unit for producing electrolyzed alkaline water and of which pH is 10 or more by electrolyzing the hypochlorite water.

The electrolyzed alkaline water production unit includes an anode cell and a cathode cell which are divided by a diaphragm and it is preferred that the electrolyzed alkaline water is generated in the cathode cell.

It is preferred that the hypochlorite water production unit generates the hypochlorite water by non-diaphragm electrolysis.

It is preferred that the hypochlorite water production unit and the electrolyzed alkaline water production unit are one same unit and neutral water in which the hypochlorite electrolysis product is solved is electrolyzed to produce the electrolyzed alkaline water having a pH value of 10 or more.

A feature of the present invention is an alkaline sterilizing water production method and includes an electrolyzed alkaline water production step in which electrolyzed alkaline water is produced by electrolyzing neutral water and a hypochlorite water production step in which the electrolyzed alkaline water, in which hypochlorite electrolysis product for generating hypochlorous acid by electrolysis is solved, is electrolyzed to produce hypochlorite water having a pH value of 10 or more.

A feature of the present invention is an alkaline sterilizing water production method and includes a hypochlorite water production step in which neutral water, in which hypochlorite electrolysis product for generating hypochlorous acid by electrolysis is solved, is electrolyzed to produce hypochlorite water and electrolyzed alkaline water production step in which the hypochlorite water is electrolyzed to produce electrolyzed alkaline water having a pH value of 10 or more.

It is preferred that the electrolyzed alkaline water is generated in the cathode cell, between anode and cathode cells divided by a diaphragm, in the electrolyzed alkaline water production step.

It is preferred that the hypochlorite water is produced by non-diaphragm electrolysis in the hypochlorite water production step.

It is preferred that the electrolyzed alkaline water production step and the hypochlorite water production step are one same step and neutral water in which the hypochlorite electrolysis product is solved is electrolyzed to produce the electrolyzed alkaline water of which pH is 10 or more.

According to the present invention, it is possible to provide an alkaline water sterilizer and a production method of alkaline sterilizing water which has both enough sterilizing effect and enough detergent effect and, in addition, has better storage stability and safety when compared with strong alkaline solution.

### THE BEST MODE FOR CARRYING OUT THE CLAIMED INVENTION

As a result of reviewing by the inventor of the present invention, surprisingly, it was found out that alkaline sterilizing water of which pH is 10 or more, preferably, 11.5 or more, has enough detergent effect. This can be assumed to be caused by effect of alkaline property having enough pH value such as, for example, emulsification effect to grease or interfacial activation effect.

Therefore, when this strong alkaline water can be acquired from neutral water, it is regarded that better storage stability and safety than using strong alkaline solution before use can be achieved and, in addition, an advantage of detergent effect of strong alkali can be achieved, too. Furthermore, when hypochlorous acid can be generated from this strong alkali, it is possible to obtain enough sterilizing effect.

Based on such a viewpoint, as a result of keen examination by the inventor of the present invention, an alkaline water sterilizer and an alkaline sterilizing water production method were invented.

### “Alkaline Water Sterilizer”

Hereafter, embodiments for realizing the invention will be explained based on figures. With regards to the present embodiments, they are embodiments for realizing the present embodiment and the present invention is not limited by the present embodiments.

In the present embodiment, because it is preferable, as an example, sodium chloride (NaCl) is used as hypochlorite electrolysis product for generating hypochlorous acid by electrolysis and tap water is used as neutral water. However, the present invention is not limited to the above. Hypochlorite electrolysis product and neutral water may be appropriately selected and used. The hypochlorite electrolysis product may be added at least before a step of generating hypochlorite water by electrolysis and adding point or timing for addition is not especially limited and may be appropriately selected.

As a hypochlorite electrolysis product, any substance which is solved by neutral water and generates hypochlorous acid ion can be appropriately selected for use. For example, it is preferable to use a substance including chlorine ion or chloride ion (Cl⁻) such as HCl (hydrochloric acid), KCl, MgCl₂, any substance including these substances, or a substance concomitantly using these substances. More preferable substance is NaCl and it has an advantage of easy acquisition, low price, safety, and the like. Such a substance including Cl⁻ generates chlorine gas by electrolytic oxidation when electrolyzed and the chlorine gas thus generated is solved in water to produce hypochlorite ion in the water. The hypochlorite electrolysis product may be a substance which directly generates hypochlorite ion by electrolysis or may be a substance which indirectly generates hypochlorite ion when electrically generated chlorine is solved as in the present embodiment.

Neutral water may be appropriately selected for use. In the present application, notion of neutrality is water having a pH value in a range between 4.5 and 8.5 and, preferably, between 5 and 8, more preferably, around 7. When tap water is used as in the present embodiment, there are advantages such as appropriateness, easiness to acquire, low price, and safety. Neutral water is not limited only to tap water and may be any of pure water, filtrate water, distilled water, groundwater, underflow water, demineralized water, purified water (RO water or membrane processed water), rain water, well water, neutralized water obtained by neutralization of acid and alkali, neutral solution including inorganic salt such as sodium bicarbonate or metallic salt or an organic matter, or water solution in which hypochlorite electrolysis product is previously solved or mixture of them. The notion of neutral water includes all of the above.

As DC power source for electrolysis which is an energy supply means used for the present embodiment, one which can be used for each purpose may be selected appropriately and there is no limitation to the power supply. It is preferable to use DC power source but an AC power source may be used as well. In the present embodiment, because it is preferable, a DC power source for electrolysis used is a constant power type power source. According to this constant power type power source, when the power is turned on, set current value is always maintained so that stable electrolysis condition can be maintained. Moreover, to the increase of resistance by scale build-up on the surface of an electrode, the power source has a function to maintain set current value by automatic voltage raising and to the decrease of current caused by deterioration of an electrode, same can be applied. For example, when the constant power set value is assumed to be 12A (constant current set value can be changed) voltage control range can be between 0 and 40V.

An electrode used in the present embodiment is not especially limited and can be appropriately selected and applied. Preferably, because the electrode is prone to oxidation as electron transfer actively occurs between electrodes by electrolysis, it is desired to coat the surface of the electrode with precious metal such as platinum or iridium. Shape of the electrode is not especially limited, too, and any form can be appropriately selected for use. Preferably, one in which an expand, a circular hole, cross-shaped hole, a square hole, or a slit-like slotted hole is shaped so that component of water solution can easily enter (ion exchange is easily occurred) is desired.

In the electrolyzer, when an electrode is configured by a plurality of electrode plates for energization, as a method of energization, there are two systems, a unipolar system and multipolar system, and either may be appropriately selected for use. Here, unipolar system is a form in which all of an electrode plate is cathode or anode while multipolar system, for example, has a structure in which a plurality of electrodes are laid over with a certain space by being isolated from each other and between an electrode plate connected to anode of a power source and an electrode plate connected to cathode of the power source, at least one electrode which is not connected to either side exists.

In the electrolyzer for producing electrolyzed alkaline water, it is preferable to provide a diaphragm to strengthen alkali property on cathode side. However, the present invention is not limited thereto and the electrolyzer may be without a diaphragm when pH value of eventual alkaline sterilizing water by electrolysis is 10 or more. In the present embodiment, a diaphragm has a property to separate positive ion and negative ion of an ion-exchange membrane or the like to anode side and cathode side by electrolysis.

For the electrolyzer which produces hypochlorite water by electrolysis, it is preferable to use an non-diaphragm electrolyzer as shown in the following embodiment. This is because, in addition to advantages such as improvement of electrolysis efficiency and long life of an electrode, when a diaphragm is provided to perform oxidation of chloride ion which generates hypochlorous acid on anode side, alkali solution is neutralized. However, when predetermined strong alkali can be maintained even when a small amount of the alkali solution is neutralized, a diaphragm may be provided to perform oxidation of chloride ion on anode side so that chlorine gas which is raw material of hypochlorite ion is generated.

### EMBODIMENT 1

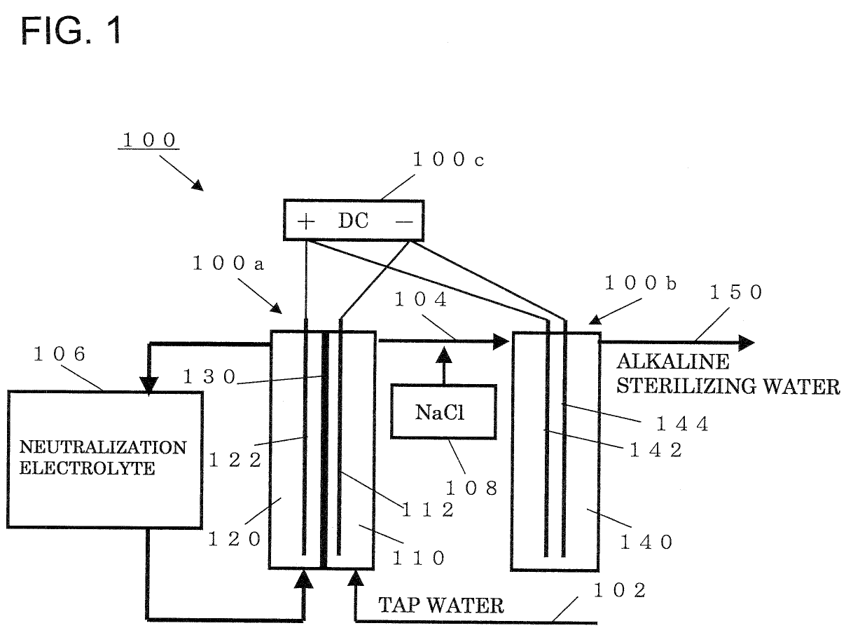

FIG. 1 shows an alkaline water sterilizer 100 according to an Embodiment 1. The alkaline water sterilizer 100 mainly includes an electrolyzed alkaline water production unit 100a for producing electrolyzed alkaline water by electrolysis, a hypochlorite water production unit 100b for producing hypochlorite water by electrolysis, and a DC power source 100c for providing DC power to these units.

The electrolyzed alkaline water production unit 10a has a shape of an electrolysis tank in which electrolysis is performed. Inside of the electrolyzed alkaline water production unit 10a is divided into a cathode cell 110 and an anode cell 120 by a diaphragm 130.

The inside of the cathode cell 110, a cathode 112 electrically connected to a cathode of the DC power is included. To the cathode cell 110, a tap water route 102 by which tap water is provided from the outside to the cathode cell 110 and a transfer route 104 for transferring electrolyzed alkaline water produced after electrolysis to the hypochlorite water production unit 100b are connected.

The inside of the anode cell 120, an anode 122 electrically connected to an anode of the DC power is included. To the anode cell 120, a neutralization circular route 106 for neutralizing acid water by providing acid water to neutralization electrolyte and/or providing the neutralization electrolyte to the acid water inside the anode cell 120 in case the inside of the anode cell 120 becomes acid is connected.

The electrolyzed alkaline water production unit 100a electrolyzes tap water provided by the tap water route 102 by the power from the DC power source 100c between the cathode 112 and the anode 122 through a diaphragm 130. As a result of electrolysis, when electrolyzed alkaline water having a pH value higher than predetermined value is produced on the cathode 110 side, through the transfer route 104, the electrolyzed alkaline water having a pH value higher than predetermined value is transferred to the hypochlorite water production unit 100b.

The hypochlorite water production unit 100b is a non-diaphragm electrolyzer. The inside of an electrolyzer 140, an anode 142 electrically connected with the anode of the DC power 100c and a cathode 144 electrically connected with the cathode of the DC power 100c are included.

To the electrolyzer 140, a transfer route 104 for providing electrolyzed alkaline water produced in the electrolyzed alkaline water production unit 100a to the cathode cell 110 and a takeoff route 150 for taking alkaline sterilizing water are connected. Here, to the transfer route 104, an addition unit 108 for adding NaCl to electrolyzed alkaline water before the water reaches the electrolyzer 140 is provided.

The hypochlorite water production unit 100b electrolyzes electrolyzed alkaline water (NaCl has already been added thereto) provided by the transfer route 104 in the electrolyzer 140 between the anode 142 and the cathode 144 by the power from the DC power source 100c by a non-diaphragm method. The electrolyzed alkaline water which generated hypochlorous acid and also became hypochlorite water (sodium hypochlorite water solution) is regarded as alkaline sterilizing water and taken from the takeoff route 150 ultimately. The alkaline sterilizing water thus taken is used for the purpose of cleansing and/or sterilizing.

In the present embodiment, it is required from the viewpoint of detergent effect that the pH value of the alkaline sterilizing water taken ultimately is 10 or more. It is especially preferable when the pH value is 11.5 or more from the viewpoint of detergent effect. Though it is required that the pH value of the alkaline sterilizing water ultimately taken is 10 or more, pH value of electrolyzed alkaline water in the middle of the process (for example electrolyzed alkaline water in the transfer route 104) is not required to be 10 or more. However, preferably, it is convenient when the pH value of electrolyzed alkaline water before entering the hypochlorite water production unit 100b is set to be 10 or more, more preferably 11.5 or more, because in that case, pH value of the alkaline sterilizing water ultimately taken can easily satisfy the condition of 10 or more or more preferably, 11.5 or more. Measuring method of pH may be appropriately selected and performed by setting a pH meter or the like and time for electrolysis may be controlled on the basis of the measuring method so that the pH value becomes a predetermined value.

### EMBODIMENT 2

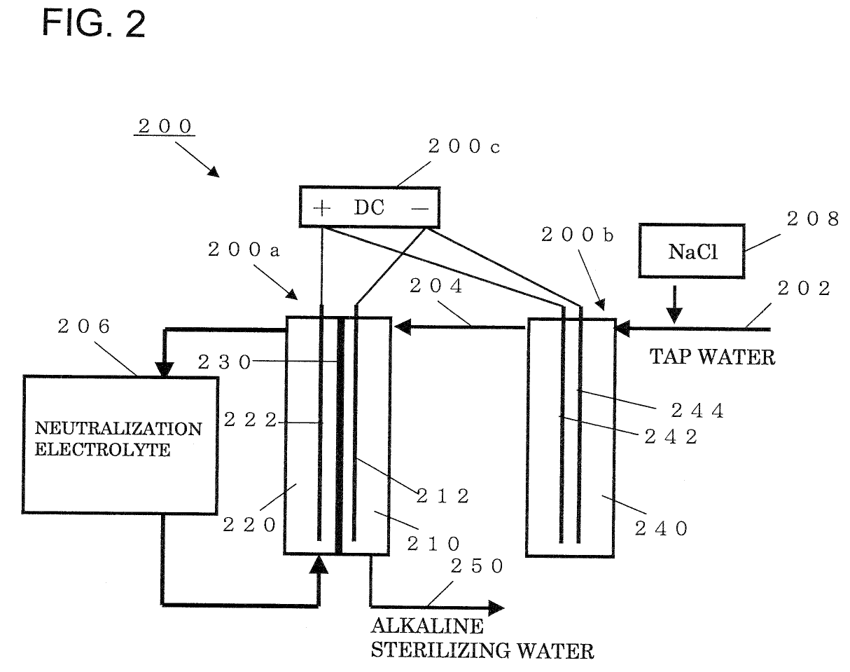

FIG. 2 shows an alkaline water sterilizer 200. The alkaline water sterilizer 200 mainly includes an electrolyzed alkaline water production unit 200a for producing electrolyzed alkaline water by electrolysis, a hypochlorite water production unit 200b for producing hypochlorite water by electrolysis, and a DC power source 200c for providing DC power to these units.

The hypochlorite water production unit 200b is an non-diaphragm electrolyzer. Inside of the electrolyzer 240, an anode 242 electrically connected with an anode of the DC power and a cathode 244 electrically connected with a cathode of the DC power are included.

To the electrolyzer 240, a tap water route 202 by which tap water is provided from outside to the electrolyzer 240 and a transfer route 204 for transferring hypochlorite water produced after electrolysis to the electrolyzed alkaline water production unit 200a are connected. Here, to the tap water route 202, an addition unit 208 for adding NaCl on the way before the water reaches the electrolyzer 240 is provided.

The hypochlorite water production unit 200b electrolyzes tap water (NaCl has already been added thereto) provided by the tap water route 202 in the electrolyzer 240 between the anode 242 and the cathode 244 by the power from the DC power source 200c by a non-diaphragm method. Due to electrolysis, hypochlorous acid is generated and also water becomes hypochlorite water (sodium hypochlorite water solution). Hypochlorite water thus produced by electrolysis is provided to the electrolyzed alkaline water production unit 200a by a transfer route 204.

The electrolyzed alkaline water production unit 200a has a shape of an electrolysis tank in which electrolysis is performed. Inside of the electrolyzed alkaline water production unit 200a is divided into a cathode cell 210 and an anode cell 220 by a diaphragm 230.

Inside of the cathode cell 210, a cathode 212 electrically connected to a cathode of the DC power source 200c is included. To the cathode cell 210, a transfer route 204 for transferring electrolyzed alkaline water produced by electrolysis in the hypochlorite water production unit 200b and a takeoff route 250 for taking alkaline sterilizing water are connected.

Inside of the anode cell 220, an anode 222 electrically connected to an anode of the DC power is included. To the anode cell 220, a neutralization circular route 206 for neutralizing acid water by providing acid water to neutralization electrolyte and/or providing the neutralization electrolyte to the acid water inside the anode cell 220 in case inside of the anode cell 220 becomes acid is connected.

The electrolyzed alkaline water production unit 200a electrolyzes hypochlorite water provided by the hypochlorite water production unit 200b by the power from the DC power source 200c between the cathode 212 and the anode 222 through the diaphragm 230. As a result of electrolysis, electrolyzed alkaline water having a pH value of 10 or more (preferably, pH value of 11.5 or more) is produced. Hypochlorite water, which satisfies this condition and also is electrolyzed alkaline water, is regarded as alkaline sterilizing water and taken out from a takeoff route 250. The alkaline sterilizing water thus taken out is used for the purpose of cleansing and/or sterilizing.

### EMBODIMENT 3

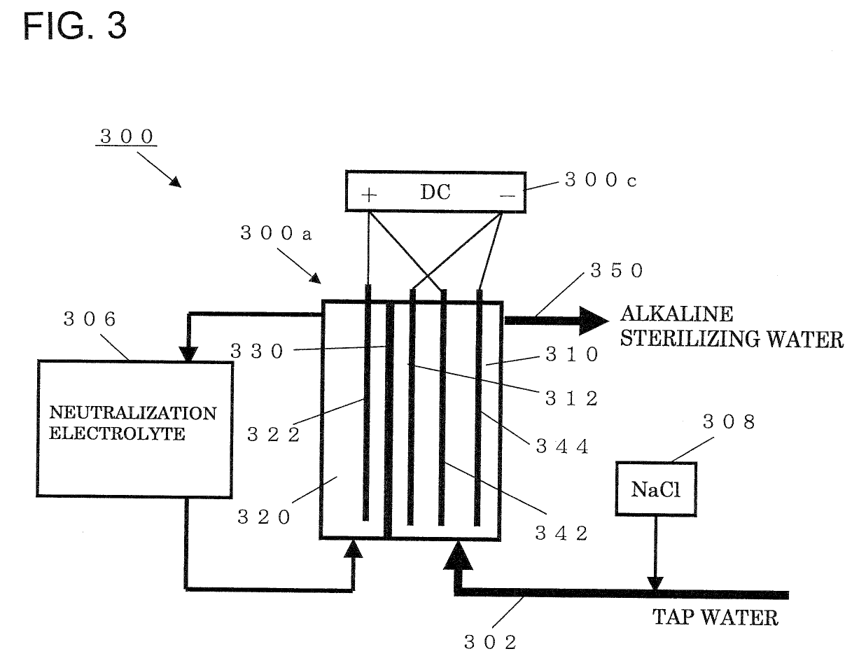

FIG. 3 shows an alkaline water sterilizer 300. The alkaline water sterilizer 300 mainly includes an electrolyzed alkaline water production unit 300a for producing electrolyzed alkaline water by electrolysis and producing hypochlorite water by electrolysis and a DC power source 300c for providing DC power to this unit.

The electrolyzed alkaline water production unit 300a has a shape of an electrolysis tank in which electrolysis is performed. The inside of the electrolyzed alkaline water production unit 300a is divided into a cathode cell 310 and an anode cell 320 by a diaphragm 330.

The inside of the cathode cell 310, a cathode 312 electrically connected to a cathode of the DC power source 300c is included. To the cathode cell 310, a tap water route 302 by which tap water is provided from the outside to the cathode cell 310 and a takeoff route 350 for taking out electrolyzed alkaline water produced after electrolysis are connected. To the tap water route 302, an addition unit 308 for adding NaCl on the way before the water reaches the cathode cell 310 is provided.

The cathode cell 310 is a non-diaphragm electrolyzer. The inside of the cathode cell 310, an anode 342 electrically connected with an anode of the DC power and a cathode 344 electrically connected with a cathode of the DC power are included.

The inside of the anode cell 320, an anode 322 electrically connected to an anode of the DC power source 300c is included. To the anode cell 320, a neutralization circular route 306 for neutralizing acid water by providing the acid water to neutralization electrolyte and/or providing the neutralization electrolyte to the acid water inside the anode cell 320 in case inside of the anode cell 320 becomes acid is connected.

The electrolyzed alkaline water production unit 300a electrolyzes tap water (NaCl has already been added thereto) provided by the tap water route 302 between the cathode 312 and the anode 322 by a diaphragm 330 to produce electrolyzed alkaline water in the cathode cell 310.

Moreover, the electrolyzed alkaline water production unit 300a electrolyzes tap water (NaCl has already been added thereto) provided by the tap water route 302 between the anode 342 and the cathode 344 by the power from the DC power source 300c by a non-diaphragm method. Hypochlorous acid is generated by electrolysis and hypochlorite water (sodium hypochlorite water solution) is produced. When pH value of electrolyzed alkaline water in the cathode cell 340 becomes 10 or more, preferably 11.5 or more, and hypochlorous acid is generated, the water is ultimately taken out from a takeoff route 350 as alkaline sterilizing water. The alkaline sterilizing water thus taken out is used for the purpose of cleansing and/or sterilizing.

According to the Embodiment 3, the hypochlorite water production unit can be provided in the same unit as the electrolyzed alkaline water production unit. Therefore, equipment can be simplified and space can be saved.

In the alkaline sterilizing water production unit according to the above embodiments, tap water is used as raw material. Therefore, strong alkali is not required to be used and it is preferable from a viewpoint of storage stability and safety. Moreover, since alkaline sterilizing water having a pH value of at least 10 or more is used, enough detergent effect can be obtained. In addition, to improve sterilizing effect which was not sufficient only with alkaline property, hypochlorite ion having sterilizing effect is included. Therefore, more sufficient sterilizing effect can be obtained.

This alkaline sterilizing water can be used for general purposes which require cleansing and sterilization and its purpose is not especially limited. For example, the water can be used for detergent and sterilization of waste plastic such as domestic garbage, detergent and sterilization of vegetable, and also detergent and sterilization of plates and utensils.

### BRIEF DESCRIPTION OF THE DRAWINGS

[FIG. 1] A schematic view for showing an alkaline water sterilizer in the first embodiment.

[FIG. 2] A schematic view for showing an alkaline water sterilizer in the second embodiment.

[FIG. 3] A schematic view for showing an alkaline water sterilizer in the third embodiment.

### DESCRIPTION OF THE REFERENCE NUMERALS

100, 200, 300 alkaline water sterilizer
100a, 200a, 300a electrolyzed alkaline water production unit
100b, 200b hypochlorite water production unit
100c, 200c, 300c DC power source

1. An alkaline water sterilizer comprising:
an electrolyzed alkaline water production unit for producing electrolyzed alkaline water by electrolyzing neutral water, and
a hypochlorite water production unit for producing hypochlorite water of which pH is 10 or more by electrolyzing the electrolyzed alkaline water in which hypochlorite electrolysis product which generates hypochlorous acid by electrolysis is solved.

2. An alkaline water sterilizer comprising:
a hypochlorite water production unit for producing hypochlorite water by electrolyzing neutral water in which hypochlorite electrolysis product which generates hypochlorous acid by electrolysis is solved, and
an electrolyzed alkaline water production unit for producing electrolyzed alkaline water of which pH is 10 or more by electrolyzing the hypochlorite water.

3. The alkaline water sterilizer according to claim 1, wherein the electrolyzed alkaline water production unit includes an anode cell and a cathode cell which are divided by a diaphragm and the electrolyzed alkaline water is generated in the cathode cell.

4. The alkaline water sterilizer according to claim 1, wherein the hypochlorite water production unit produces the hypochlorite water by non-diaphragm electrolysis.

5. The alkaline water sterilizer according to claim 1, wherein the electrolyzed alkaline water production unit and the hypochlorite water production unit are the same unit and neutral water in which hypochlorite electrolysis product is solved is electrolyzed to produce the electrolyzed alkaline water having a pH value of 10 or more.

6. An alkaline sterilizing water production method comprising:
an electrolyzed alkaline water production step in which electrolyzed alkaline water is produced by electrolyzing neutral water and;
a hypochlorite water production step in which the electrolyzed alkaline water, in which hypochlorite electrolysis product for generating hypochlorous acid by electrolysis is solved, is electrolyzed to produce hypochlorite water having a pH value of 10 or more.

7. An alkaline sterilizing water production method comprising:
a hypochlorite water production step in which neutral water, in which hypochlorite electrolysis product for producing hypochlorous acid by electrolysis is solved, is electrolyzed to produce hypochlorite water and;
an electrolyzed alkaline water production step in which the hypochlorite water is electrolyzed to produce electrolyzed alkaline water having a pH value of 10 or more.

8. The alkaline sterilizing water production method according to claim 6, wherein the electrolyzed alkaline water is produced in the cathode cell between anode and cathode cells divided by a diaphragm in the electrolyzed alkaline water production step.

9. The alkaline sterilizing water production method according to claim 6, wherein the hypochlorite water is produced by non-diaphragm electrolysis in the hypochlorite water production step.

10. The alkaline sterilizing water production method according to claim 6, wherein the electrolyzed alkaline water production step and the hypochlorite water production step are the same step and neutral water in which the hypochlorite electrolysis product is solved is electrolyzed to produce the electrolyzed alkaline water having a pH value of 10 or more.

11. The alkaline water sterilizer according to claim 2, wherein the electrolyzed alkaline water production unit includes an anode cell and a cathode cell which are divided by a diaphragm and the electrolyzed alkaline water is generated in the cathode cell.

12. The alkaline water sterilizer according to claim 2, wherein the hypochlorite water production unit produces the hypochlorite water by non-diaphragm electrolysis.

13. The alkaline water sterilizer according to claim 2, wherein the electrolyzed alkaline water production unit and the hypochlorite water production unit are the same unit and neutral water in which hypochlorite electrolysis product is solved is electrolyzed to produce the electrolyzed alkaline water having a pH value of 10 or more.

14. The alkaline sterilizing water production method according to claim 7, wherein the electrolyzed alkaline water is produced in the cathode cell between anode and cathode cells divided by a diaphragm in the electrolyzed alkaline water production step.

15. The alkaline sterilizing water production method according to claim 7, wherein the hypochlorite water is produced by non-diaphragm electrolysis in the hypochlorite water production step.

16. The alkaline sterilizing water production method according to claim 7, wherein the electrolyzed alkaline water production step and the hypochlorite water production step are the same step and neutral water in which the hypochlorite electrolysis product is solved is electrolyzed to produce the electrolyzed alkaline water having a pH value of 10 or more.
